(19) United States
(12) Patent Application Publication
(10) Pub. No.: US 2025/0049380 A1
(43) Pub. Date: Feb. 13, 2025

(54) MULTI-ELECTRODE PATCH FOR MYOTRACE MEASUREMENT

(71) Applicant: KONINKLIJKE PHILIPS N.V., EINDHOVEN (NL)

(72) Inventors: PASCAL DE GRAAF, EINDHOVEN (NL); OTTO PAULUS HENDRIKUS KOKKE, VLEUTEN (NL)

(21) Appl. No.: 18/720,819

(22) PCT Filed: Dec. 16, 2022
(86) PCT No.: PCT/EP2022/086331

§ 371 Date: Jun. 17, 2024

Related U.S. Application Data
(60) Provisional application No. 63/290,880, filed on Dec. 17, 2021.
(60) Provisional application No. 63/298,715, filed on Jan. 12, 2022.

(51) Int. Cl. A61B 5/397; A61B 5/00; A61B 5/085; A61B 5/259; A61B 5/266; A61B 5/296; A61B 5/313

(52) U.S. Cl. CPC A61B 5/397; A61B 5/085; A61B 5/259; A61B 5/266; A61B 5/296; A61B 5/313; A61B 5/6823; A61B 2562/046; A61B 2562/222; A61B 2562/227

(57) ABSTRACT

A streamlined system for non-invasively determining a neural respiratory drive (NRD) index of a patient includes an EMG patch that provides three integrated EMG electrodes: two signal EMG electrodes to be positioned in the second intercostal space on either side of the sternum and one reference EMG electrode to be positioned on the sternum above the two signal electrodes. The integration of all three EMG electrodes in a single patch enables clinicians to spend less time determining where to place the patch than they would if they had to determine the proper positioning of three single EMG electrodes. In addition, the disclosed system provides a single cable that can electrically connect to all three EMG electrodes when inserted into a plug on the EMG patch, thereby reducing cable clutter that would otherwise result from having to provide a separate cable for each EMG electrode.

### BACKGROUND OF THE INVENTION

### 1. Field of the Invention

The disclosed concept pertains to systems for quantifying breathing effort of patients and, in particular, to systems that monitor the neuro muscular drive (NMD) of patients.

### 2. Description of the Related Art

Electromyography (EMG) can be used to non-invasively assess the respiratory status of a patient by monitoring the activity of muscles involved in respiration, such as the intercostal spaces on bilateral sides of the sternum (parasternal) or the abdominal area close to the diaphragm. EMG measurements of inspiratory muscle activity are indicators of the balance between respiratory muscle load and respiratory muscle capacity, and can be used to obtain an objective measure of breathing effort. In particular, respiratory EMG activity as measured during inhalations can be associated with the neural respiratory drive (NRD), which is a signal that the brain outputs to the respiratory muscles and an indicator of the balance between respiratory muscle load and respiratory muscle capacity.

Objective measures of respiratory muscle activity derived from EMG signals are considered to be important for monitoring the respiratory status of patients, such as in patients with chronic obstructive pulmonary disease (COPD). While respiration rate is easily and non-invasively determined, respiration rate does not indicate how much effort a patient expends to breathe. For example, if a COPD patient and a relatively healthy person were to breathe at the same rate, it is understood that the COPD patient would expend more effort than the healthy person to breathe at that rate, but the respiration rate alone would not provide an indication of how much effort each person exerts to breathe at that rate. In contrast, EMG activity of respiratory muscles can be used to calculate NRD and provide an objective quantification of the effort required for a given patient to breathe at a particular rate, but EMG activity needs to be monitored at a few locations on the chest of the patient, requiring that several EMG electrodes and cables be properly positioned and connected to a monitor. It will be appreciated that clinicians are often very busy, and every minute spent on working to properly set up equipment for NRD index calculation is time that does not get spent on direct patient interaction.

Accordingly, there is room for improvement in systems used to monitor neural respiratory drive.

### SUMMARY OF THE INVENTION

Accordingly, it is an object of the present invention to provide a streamlined system for non-invasively determining a neural respiratory drive (NRD) index of a patient using a single EMG patch that includes three integrated EMG electrodes: two signal EMG electrodes to be positioned in the second intercostal space on either side of the sternum and one reference EMG electrode to be positioned on the sternum above the two signal electrodes. The patch includes connector hardware comprising a socket structured to receive the plug of a single cable that is structured to electrically connect to all three EMG electrodes when inserted into the plug, thereby reducing cable clutter that would otherwise result from having to provide a separate cable for each EMG electrode in a traditional EMG system.

In one embodiment, an EMG patch for use with a neural respiratory drive monitoring system is structured to be adhered to the chest of a patient and includes: an adhesive layer comprising a plurality of cutouts and structured to removably adhere the patch to the chest of the patient; a plurality of electrolyte gel pads equal in number to the plurality of cutouts, each gel pad being inserted within and filling a corresponding one of the plurality of cutouts; circuitry coupled to the adhesive layer and structured to sense EMG signal activity during respiratory activity of the patient through each of the plurality of electrolyte gel pads; and connection hardware coupled to and electrically connected to the circuitry, the connection hardware comprising a socket. The patch is structured such that placing the patch on the chest of the patient for EMG monitoring disposes a first of the plurality of cutouts on a second intercostal space on a first side of the sternum of the patient, disposes a second of the plurality of cutouts on a second intercostal space on a second side of the sternum of the patient, and disposes a third of the plurality of cutouts on the sternum of the patient. The socket is configured to receive a single cable structured to transmit all EMG signal activity sensed by the circuitry to a controller.

In another embodiment, a system for monitoring neural respiratory drive of a patient during respiration includes: a controller configured to calculate a neural respiratory drive index based on received EMG signal activity, a single cable electrically connected to the controller, an EMG patch structured to be adhered to the chest of the patient, and connection hardware. The EMG patch includes: an adhesive layer comprising a plurality of cutouts and structured to removably adhere the patch to the chest of the patient; a plurality of electrolyte gel pads equal in number to the plurality of cutouts, each gel pad being inserted within and filling a corresponding one of the plurality of cutouts; and circuitry coupled to the adhesive layer and structured to sense EMG signal activity during respiratory activity of the patient through each of the plurality of electrolyte gel pads. The connection hardware includes a socket coupled to the circuitry, and a plug coupled to and electrically connected to the cable, and structured to be coupled to the socket. The patch is structured such that placing the patch on the chest of the patient for EMG monitoring disposes a first of the plurality of cutouts on a second intercostal space on a first side of the sternum of the patient, disposes a second of the plurality of cutouts on a second intercostal space on a second side of the sternum of the patient, and disposes a third of the plurality of cutouts on the sternum of the patient. The cable is structured to transmit all EMG signal activity sensed by the circuitry to the controller.

A system for monitoring neural respiratory drive of a patient during respiration includes: a single cable structured to electrically connect to a controller, an EMG patch, and connection hardware. The EMG patch includes: an adhesive layer that is structured to removably adhere the patch to the chest of the patient and includes a plurality of electrolyte gel pad inserts, and circuitry that is coupled to the adhesive layer and structured to sense EMG signal activity during respiratory activity of the patient through each of the plurality of electrolyte gel pad inserts. The connection hardware includes a socket coupled to the circuitry, as well as a plug coupled to and electrically connected to the cable and structured to be coupled to the socket. The patch is structured such that placing the patch on the chest of the patient for EMG monitoring positions the circuitry so as to be able to sense EMG signals that can be used to calculate the neural respiratory drive index. The cable is structured to transmit all EMG signal activity sensed by the circuitry to the controller.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 shows a system representative of known systems used to sense monitor EMG activity during respiration;

FIG. 2 shows a neural respiratory drive (NRD) monitoring system, in accordance with exemplary embodiments of the present invention;

FIG. 3A shows an elevation view of an adhesive backing layer of an EMG patch used with the NRD monitoring system shown in FIG. 2, in accordance with exemplary embodiments of the present invention;

FIG. 3B shows an elevation view of an adhesive layer of the EMG patch shown in FIG. 2, in accordance with exemplary embodiments of the present invention;

FIG. 3C shows cutouts in the adhesive layer shown in FIG. 3B filled in with electrolyte gel pads, in accordance with exemplary embodiments of the present invention;

FIG. 3D shows EMG sensing circuitry coupled to the arrangement shown in FIG. 3C, in accordance with exemplary embodiments of the present invention;

FIG. 3E shows an insulative hardware backing coupled to the arrangement shown in FIG. 3D, in accordance with exemplary embodiments of the present invention;

FIG. 3F shows connective hardware coupled to the arrangement shown in FIG. 3E, in accordance with exemplary embodiments of the present invention;

FIG. 4A an elevation view of a socket component of the connector hardware shown in FIG. 3F, in accordance with exemplary embodiments of the present invention;

FIG. 4B shows a sectional view of the socket component along the line 4B-4B shown in FIG. 4A;

FIG. 4C shows an elevation view of a cable plug component of the connector hardware shown in FIG. 3F, with a portion of the housing removed in order to show the interior components, in accordance with exemplary embodiments of the present invention;

FIG. 4D shows an elevation view of the cable plug component shown in FIG. 4C, with the housing intact, in accordance with exemplary embodiments of the present invention; and

FIG. 4E shows a sectional view of the cable plug component along the line 4E-4E shown in FIG. 4D.

### DETAILED DESCRIPTION OF EXEMPLARY EMBODIMENTS

As used herein, the singular form of “a”, “an”, and “the” include plural references unless the context clearly dictates otherwise.

As used herein, the statement that two or more parts or components are “coupled” shall mean that the parts are joined or operate together either directly or indirectly, i.e., through one or more intermediate parts or components, so long as a link occurs.

As used herein, the term “controller” shall mean a number of programmable analog and/or digital devices (including an associated memory part or portion) that can store, retrieve, execute and process data (e.g., software routines and/or information used by such routines), including, without limitation, a field programmable gate array (FPGA), a complex programmable logic device (CPLD), a programmable system on a chip (PSOC), an application specific integrated circuit (ASIC), a microprocessor, a microcontroller, a programmable logic controller, or any other suitable processing device or apparatus. The memory portion can be any one or more of a variety of types of internal and/or external storage media such as, without limitation, RAM, ROM, EPROM(s), EEPROM(s), FLASH, and the like that provide a storage register, i.e., a non-transitory machine readable medium, for data and program code storage such as in the fashion of an internal storage area of a computer, and can be volatile memory or nonvolatile memory.

As used herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

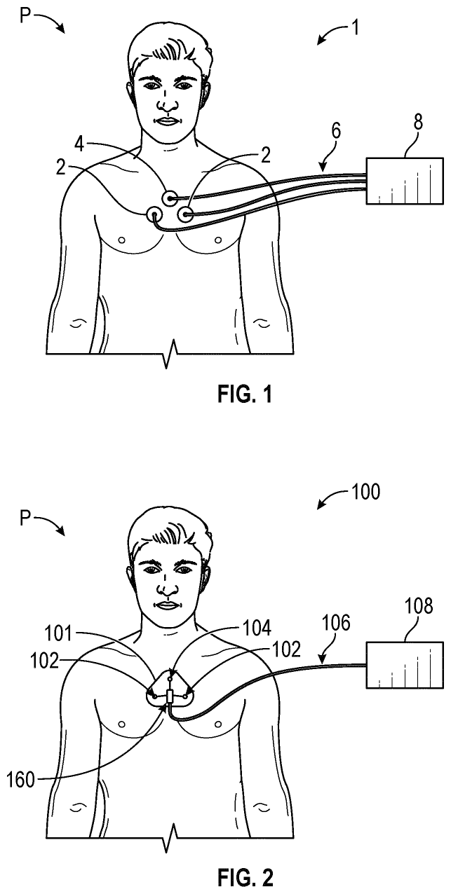

The present invention, as described in greater detail herein in connection with various particular exemplary embodiments, provides streamlined systems for non-invasively determining a neural respiratory drive (NRD) index of a patient. NRD is considered an objective quantification of breathing effort, and quantifying NRD non-invasively requires using EMG electrodes to detect respiratory muscle activity during inspiration, as EMG measurements taken during inhalation are considered to be indicators of the balance between respiratory muscle load and respiratory muscle capacity. As detailed herein below with respect to FIG. 1, the optimal EMG setup for determining an NRD index requires placing three EMG electrodes at precise locations on the chest of a patient, with precise distancing between the electrodes, and it will be appreciated that the precise locations and distancing need to be re-determined every time that the electrodes need to be replaced. In addition, electrical leads need to be snapped onto all three electrodes, and three different cables (one for each electrode) need to be connected to the leads. Depending on the mechanism available for connecting the cables to the leads, connecting the cables to the leads may require applying undesirable pressure to the patient's chest. The systems disclosed herein and shown in FIGS. 2, 3A-3F, and 4A-4E provide a streamlined EMG monitoring setup to be used for determining NRD index that avoids the disadvantages iterated above.

Referring now to FIG. 1, a system 1 representative of known systems for sensing inspiratory EMG signals is shown. Determining the NRD index for a patient P using known equipment such as that equipment shown in system 1 requires placing two signal EMG electrodes 2 and one reference EMG electrode 4 on the upper chest of patient P. One signal EMG electrode 2 is placed on one side of the sternum in the second intercostal space, the second signal EMG electrode 2 is placed on the other side of the sternum in the second intercostal space, and the reference EMG electrode 4 is placed on the sternum, above (relative to the view shown in FIG. 1) the two signal electrodes 2. It should be noted that, when the term “above” is used herein in reference to two or more components (e.g. electrodes) positioned on the chest of patient P, “above” denotes being closer to the neck of patient P such that, if a first component is disposed above a second component on the chest of patient P, then the first component is disposed closer to the neck of patient P than the second component.

In FIG. 1, each electrode 2,4 is connected by its own designated cable 6 to a controller 8. Three different leads (not numbered in FIG. 1), one for each electrode 2,4, needs to be snapped onto each electrode in order to provide a connection point for each cable 6 to connect to on the electrode 2,4. It will be appreciated that decreasing the time it takes to properly position the three separate electrodes 2,4, eliminating the need to apply pressure to patient P′s chest in order to snap a lead onto each electrode 2,4, and reducing cable clutter created by having three separate cables 6 is desirable.

Referring now to FIG. 2, a NRD monitoring system 100 according to an exemplary embodiment of the present invention shown. Rather than including three separate EMG electrodes as system 1 in FIG. 1 does, system 100 in FIG. 2 comprises a single EMG patch 101 that comprises two EMG signal electrodes 102 and one reference EMG electrode 104 integrated into the patch 101. In an exemplary embodiment, patch 101 is produced to be disposable. System 100 further comprises a single cable 106 that is structured to be coupled to patch 101 at a first end and to be coupled to a controller 108 at a second end disposed opposite the first end, rather than using an individual cable for each electrode, as known systems such as system 1 do. Coupling cable 106 to patch 101 and controller 108 establishes electrical communication between patch 101 and controller 108, and it will be appreciated that controller 108 is configured to calculate the NRD index using the EMG signals sensed by patch 101 after the signals have been transmitted to controller 108 via cable 106.

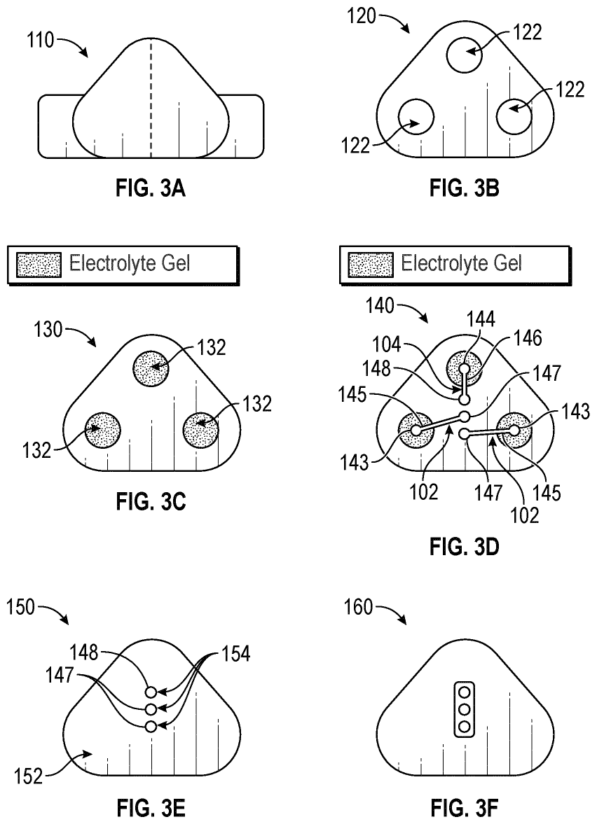

Referring now to FIGS. 3A-3F, elevation views of each of the layers used to construct patch 101 shown in FIG. 2 are shown. These layers, which are described in further detail herein below, include an adhesive backing 110, an adhesive layer 120, a gel layer 130, a circuitry layer 140, a hardware backing 150, and a connector hardware layer 160. Each layer depicted in FIGS. 3A-3F is substantially flat and comprises two sides, one side being a chest-facing side, and the other side being an outward-facing side disposed opposite the chest-facing side. The term “chest-facing”, as used herein with respect to patch 101 and any components thereof, should be understood to be indicative of facing toward the chest of patient P when patch 101 is placed on the chest of patient P. Conversely, the term “outward”, as used herein with respect to patch 101 and any components thereof, should be understood to be indicative of facing away from the body of patient P rather than toward the chest of patient P when patch 101 is placed on the chest of patient P.

It should be noted that only the outward-facing side of each layer is shown in FIGS. 3B-3F, and that FIG. 3A is an adhesive backing that is removed before patch 101 is placed on patient P for use in NRD monitoring. Before each of the layers 110, 120, 130, 140, 150, and 160 are described in detail, it should be noted that patch 101 is assembled by coupling the chest-facing side of adhesive layer 120 to the outward-facing side of adhesive backing 110, coupling the chest-facing side of gel layer 130 to the outward-facing side of adhesive layer 120, coupling the chest-facing side of circuitry layer 140 to the outward-facing side of gel layer 130, coupling the chest-facing side of hardware backing 150 to the outward-facing side of circuitry layer 140, and coupling the chest-facing side of connector hardware 160 to the outward-facing side of hardware backing 150.

Referring now to FIGS. 3A and 3B, adhesive backing 110 and adhesive layer 120 are shown. Adhesive backing 110 and adhesive layer 120 are of the type often found on self-adhering patches. Adhesive backing 110 is structured to preserve the adhesive quality of adhesive layer 120 prior to use of patch 101 and can comprise, for example and without limitation, two pieces of thin, flexible plastic or waxed paper that are placed on the chest-facing side of adhesive 120 during the assembly of patch 101, and are structured to be pulled away to expose the adhesive 120 when it is time to adhere patch 101 to the skin of patient P. Adhesive layer 120 is formed with cutouts 122 and can comprise any type of material suitable for adhering a wearable patch to skin, including, for example and without limitation, polymer glue or silicone. In FIG. 3C, gel layer 130 is shown. Gel layer 130 comprises three separate electrolyte gel pads 132 structured to be inserted within and fill the cutouts 122 in adhesive layer 120 such that a chest facing side of each gel pad 132 lies against the skin on the chest of patient P when the patch 101 is placed on patient P.

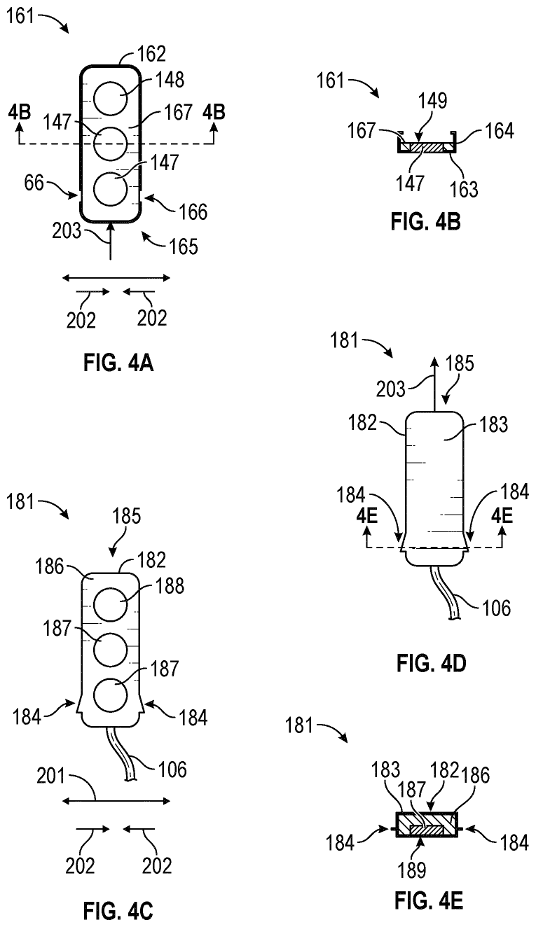

In FIG. 3D circuitry layer 140 is shown. Circuitry layer 140 comprises two signal electrodes 102 and a reference electrode 104 (also shown in FIG. 2). Both signal electrodes 102 comprise a signal sensing terminal 143 connected to a conductor 145 connected to a signal transmission terminal 147, and reference electrode portion similarly comprises a skin conducting terminal 144 connected to a conductor 146 connected to a reference transmission terminal 148. In an exemplary embodiment of the present invention, each signal electrode 102 and reference electrode 104 are all formed as unitary bodies, i.e. the signal sensing terminals, conductors, and communication terminals simply denote a specific region of each electrode 102 or 104. Signal sensing terminals 143,144 of each electrode 102,104 are configured to directly sense EMG activity from the chest of patient P, and it will be appreciated that circuitry layer 140, gel layer 130, and adhesive layer 120 are structured such that, when layers 120, 130, and 140 are coupled to one another, signal sensing terminals 143,144 of circuitry layer 140 align with electrolyte gel pads 132 of gel layer 130, as well as with cutouts 122 in adhesive 120, in order to allow signal sensing terminals 143,144 to sense EMG signal activity from the skin of patient P via the electrolyte gel pads 132. Conductors 145,146 conduct the signals sensed by signal sensing terminals 143,144 to the transmission terminals 147,148 so that the signals can be transmitted to controller 108 from transmission terminals 147,148, as detailed further herein with respect to FIGS. 4A-4D. As shown in FIG. 3D, both signal electrodes 102 and reference electrode 104 are electrically isolated from one another.

Hardware backing layer 150 shown in FIG. 3E comprises an insulating backing 152 formed with three cutouts 154, and is structured to shield all circuitry of circuitry layer 140 except for the transmission terminals 147,148 from the environment disposed outward relative to hardware backing layer 150. It will be appreciated that, in FIG. 3E, terminals 147,148 are shown filling the space provided by cutouts 154, and that cutouts 154 enable the connector hardware 160 shown in FIG. 3F and FIGS. 4A-4E (detailed further herein below) to electrically connect to transmission terminals 147,148 in order to transmit the sensed EMG signals to controller 108 (FIG. 2).

Referring now to FIGS. 4A, 4B, 4C, 4D, and 4E, several views of the components of connector hardware 160 are shown to reveal the various features of connector hardware 160 in detail. As stated above, connector hardware 160 comprises two main components, a socket 161 (shown in FIG. 4A) and a plug 181 (shown in FIG. 4C). These two main components are structured to be removably coupled to one another via a sliding motion (detailed further herein below). Socket 161 is also referred to herein as the patch side 161 of connector hardware 160, since it is the component of connector hardware 160 that is coupled directly to hardware backing layer 150 of patch 101. Plug 181 is also referred to herein as the cable side 181 of connector hardware 160, since it includes cable 106 (shown in FIG. 2) that transmits signals from patch 101 to controller 108. It should be noted that, with respect to FIGS. 4A-4F, the terms “lateral direction[s]” and “laterally” refer to the direction[s] indicated by arrows 201 in FIGS. 4A and 4C. In addition, it should be noted that the terms “inward”, “inward direction”, and “inwardly”, with respect to FIGS. 4A-4F, refer to the direction[s] indicated by arrows 202 in FIGS. 4A and 4C.

Referring now to FIG. 4A and FIG. 4B, socket/patch side 161 is shown in detail. FIG. 4A is an elevation view of socket 161, and FIG. 4B is a section view of socket 161 along the line 4B-4B shown in FIG. 4A. As shown in FIG. 4A, socket 161 comprises a socket housing portion 162. As shown in FIG. 4B, socket housing portion 162 comprises both a floor 163 and wall portions 164, and it should be noted that floor 163 is directly coupled to hardware backing layer 150 when patch 101 is fully assembled. Floor 163 is formed with gaps structured to receive transmission terminals 147,148 such that transmission terminals 147,148 extend outwardly relative to floor 163. It will be appreciated that side 149 of signal transmission terminal 147 shown in FIG. 4B is an outward-facing side of terminal 147.

Referring again to FIG. 4A, it should be noted that there are sections of socket housing portion 162 where gaps are formed in wall 164. These gaps include a plug receiving opening 165 and lateral openings 166. These openings 165,166 allow plug/cable side 181 to slide into socket/patch side 161, as detailed later herein with respect to FIGS. 4C-4E. Lastly, socket 161 comprises insulating material 167, configured to electrically isolate transmission terminals 147,148 from one another.

IN FIGS. 4C-4E, plug/cable side 181 of the connector hardware 160 is shown in detail. As shown in FIGS. 4C-4E, plug 181 comprises a plug housing 182. FIG. 4C shows an elevation view of cable side 181 with an outward facing portion 183 of the plug housing 182 removed (outward facing portion 183 is shown numbered in FIGS. 4D and 4E) in order to show the components housed within the interior of plug 181, while FIG. 4D shows an elevation view of plug 181 with plug housing 182 intact, i.e. with outward facing portion 183 included, as plug housing 182 is actually produced. FIG. 4E is a section view of plug 181 along the line 4E-4E shown in FIG. 4D.

As shown in FIGS. 4C-4E, plug housing 182 is formed with two flared portions 184, one formed on a first lateral side of housing 182 and the other formed on a second lateral side of housing 182 disposed opposite the first side. Referring to FIGS. 4A and 4D, it will be appreciated that plug 181 is structured to be inserted into socket 161 by inserting a top end 185 of plug 181 into plug receiving opening 165 of socket 161, as indicated by arrows 203 in FIGS. 4A and 4D. In an exemplary embodiment, the user will know that plug 181 is properly inserted into socket 161 due to a click that will be felt when flared portions 184 expand laterally (i.e. in the direction indicated by arrow 201) from the interior of socket housing portion 162 to the exterior of socket housing portion 162 through lateral openings 166. It should be noted that top end 185 is disposed opposite of cable 106 (also shown in FIG. 2). Flared portions 184 and lateral openings 166 form a slide mechanism that enables plug 181 to be inserted into socket 161 and prevents plug 181 from sliding out of socket 161 unless flared portions 184 are squeezed inward (i.e. in the direction indicated by arrows 202 in FIGS. 4A and 4C) toward one another such that they no longer extend through lateral openings 166 to the exterior of socket housing portion 162. Plug housing 182 can, for example and without limitation, be produced from any type of semi-flexible polymer that enables flared portions 184 to be pushed inward toward one another if portions 184 are squeezed toward one another with enough force. It will be appreciated that squeezing portions 184 inward toward one another enables plug 181 to be removed from socket 161 by pulling plug 181 away from socket 161 in the direction opposite of that which is indicated by arrows 203.

In FIG. 4C, two cable signal terminals 187 and one cable reference terminal 188 are shown. Terminals 187,188 are electrically connected to cable 106. It should be noted that an outward facing side of plug 181 is shown in FIG. 4C, and that cable 106 is electrically connected to terminals 187,188 via connections on a chest-facing side of plug 181 that is disposed opposite of the outward facing side and not visible in the figures. In an exemplary embodiment, cable 106 comprises three-core medical grade wiring. Plug 181 is structured such that, when plug 181 is fully inserted into socket 161, each of the two cable signal terminals 187 aligns with and electrically connects to a corresponding signal transmission terminal 147 of socket 161 and the cable reference terminal 188 aligns with and electrically connects to reference transmission terminal 148 of socket 161. Specifically, a chest-facing side of each cable terminal 187,188 physically contacts an outward facing side of the corresponding socket transmission terminal 147,188 (thus enabling electrical communication between corresponding cable terminals 187,188 and socket transmission terminals 147,148). For example, when plug 181 is fully inserted into socket 161, chest-facing side 189 (numbered in FIG. 4E) of signal terminal 187 contacts outward-facing side 149 of transmission terminal 147 shown in FIG. 4B.

The advantages of NRD monitoring system 100 over traditional EMG monitoring setups such as system 1 are abundant. The integration of both signal EMG electrodes 102 and the one reference electrode 104 into the single patch 101 significantly decreases both the time and effort it would otherwise take to properly position both signal electrodes 102 and the one reference electrode 104 in order to optimally determine NRD index. Obtaining high quality EMG signal data for calculation of NRD index requires accurately placing both signal electrodes 102 on the second intercostal spaces on either side of the sternum and the reference electrode 104 on the sternum, and ensuring that all three electrodes are properly spaced apart from one another. Patch 101 greatly reduces the effort and time required to properly position and space EMG electrodes 102,104 apart, as electrodes 102,104 are already spaced the correct distances apart on patch 101, such that a care provider only needs to determine where the second intercostal spaces on either side of the sternum are in order place patch 101 on the chest of patient P such that signal electrodes 102 are positioned on the second intercostal spaces. I.e. a care provider using patch 101 no longer needs to determine the distance from the intercostal spaces to the sternum to determine where to place reference electrode 104 relative to the signal electrodes 102, and no longer needs to correctly space the two signal electrodes 102 apart from one another in the second intercostal spaces on either side of the sternum. In addition, providing a single cable 106 that is structured to electrically connect to all three electrodes 102, 104 instead of the three separate cables 6 used for each electrode 2,4 in system 1 significantly reduces cable clutter, enhancing ease of use of system 100. Furthermore, the slide connection design of flared portions 184 on plug 181 and lateral openings 166 in socket 161 also enables patch 101 and cable 106 to be connected with a low insertion force and with virtually no pressure applied to the chest of patient P. Lastly, patch 101 can be produced relatively inexpensively and is designed to be disposable, as patch 101 contains no active electronic and cannot be disassembled, aside from adhesive backing 110 being structured to be removed from adhesive layer 120.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. An EMG patch for use with a neural respiratory drive monitoring system, the EMG patch comprising:
an adhesive layer comprising a plurality of cutouts and structured to removably adhere the patch to the chest of the patient,
a plurality of electrolyte gel pads equal in number to the plurality of cutouts, each gel pad being inserted within and filling a corresponding one of the plurality of cutouts;
circuitry coupled to the adhesive layer and structured to sense EMG signal activity during respiratory activity of the patient through each of the plurality of electrolyte gel pads; and
connection hardware coupled to and electrically connected to the circuitry, the connection hardware comprising a socket,
wherein the patch is structured such that placing the patch on the chest of the patient for EMG monitoring disposes a first of the plurality of cutouts on a second intercostal space on a first side of the sternum of the patient, disposes a second of the plurality of cutouts on a second intercostal space on a second side of the sternum of the patient, and disposes a third of the plurality of cutouts on the sternum of the patient,
wherein the socket is configured to receive a single cable structured to transmit all EMG signal activity sensed by the circuitry to a controller.

2. The EMG patch of claim 1,
wherein the plurality of cutouts comprises three cutouts and wherein the plurality of electrolyte gel pads comprises three electrolyte gel pads.

3. The EMG patch of claim 1,
wherein the circuitry comprises a plurality of electrodes,
wherein each of the plurality of electrodes is electrically isolated from every other one of the plurality of electrodes, and
wherein each of the plurality of electrodes is coupled to the socket.

4. The EMG patch of claim 3,
wherein each of the plurality of electrodes comprises a signal sensing terminal electrically connected to a transmission terminal via a conductor, and
wherein each signal sensing terminal is coupled to a corresponding one of the plurality of electrolyte gel pads and each transmission terminal is coupled to the socket.

5. The EMG patch of claim 1,
wherein the socket is configured to receive the cable via a slide mechanism.

6. A system for monitoring neural respiratory drive of a patient during respiration, the monitoring system comprising:
a controller configured to calculate a neural respiratory drive index based on received EMG signal activity;
a single cable electrically connected to the controller;
an EMG patch, the EMG patch comprising:
an adhesive layer comprising a plurality of cutouts and structured to removably adhere the patch to the chest of the patient;
a plurality of electrolyte gel pads equal in number to the plurality of cutouts, each gel pad being inserted within and filling a corresponding one of the plurality of cutouts; and
circuitry coupled to the adhesive layer and structured to sense EMG signal activity during respiratory activity of the patient through each of the plurality of electrolyte gel pads; and
connection hardware, the connection hardware comprising:
a socket coupled to the circuitry; and
a plug coupled to and electrically connected to the cable, and structured to be coupled to the socket;
wherein the patch is structured such that placing the patch on the chest of the patient for EMG monitoring disposes a first of the plurality of cutouts on a second intercostal space on a first side of the sternum of the patient, disposes a second of the plurality of cutouts on a second intercostal space on a second side of the sternum of the patient, and disposes a third of the plurality of cutouts on the sternum of the patient,
wherein the cable is structured to transmit all EMG signal activity sensed by the circuitry to the controller.

7. The monitoring system of claim 6,
wherein the plurality of cutouts comprises three cutouts and wherein the plurality of electrolyte gel pads comprises three electrolyte gel pads.

8. The monitoring system of claim 6,
wherein the circuitry comprises a plurality of electrodes,
wherein each of the plurality of electrodes is electrically isolated from every other one of the plurality of electrodes, and
wherein each of the plurality of electrodes is coupled to the socket.

9. The monitoring system of claim 8,
wherein each of the plurality of electrodes comprises a signal sensing terminal electrically connected to a transmission terminal via a conductor, and
wherein each signal sensing terminal is coupled to a corresponding one of the plurality of electrolyte gel pads and each transmission terminal is coupled to the socket.

10. The monitoring system of claim 6,
wherein the socket is configured to receive the plug via a slide mechanism.

11. The monitoring system of claim 8,
wherein the plug comprises a plurality of cable terminals corresponding in number to the plurality of electrodes, and
wherein the socket and plug are structured such that, when the socket receives the plug, each of the plurality of cable terminals is electrically connected to a corresponding one of the plurality of electrodes.

12. The monitoring system of claim 10,
wherein the socket comprises a socket housing with socket walls,
wherein the plug comprises a plug housing,
wherein the slide mechanism comprises lateral openings formed in the socket walls and flared portions formed in the plug housing,
wherein the flared portions extend away from an interior of the plug housing and are structured to extend through the lateral openings to an exterior of the socket housing when the plug is inserted into the socket, and
wherein the flared portions and lateral openings are structured such that, if the plug is inserted into the socket, force must be exerted on the flared portions inwardly relative to the plug housing in order to remove the plug from the socket.

13. A system for monitoring neural respiratory drive of a patient during respiration, the monitoring system comprising:
a single cable structured to electrically connect to a controller;
an EMG patch, the EMG patch comprising:
an adhesive layer structured to removably adhere the patch to the chest of the patient and comprising a plurality of electrolyte gel pad inserts; and
circuitry coupled to the adhesive layer and structured to sense EMG signal activity during respiratory activity of the patient through each of the plurality of electrolyte gel pad inserts; and
connection hardware, the connection hardware comprising:
a socket coupled to the circuitry; and
a plug coupled to and electrically connected to the cable, and structured to be coupled to the socket;
wherein the patch is structured such that placing the patch on the chest of the patient for EMG monitoring positions the circuitry so as to be able to sense EMG signals that can be used to calculate the neural respiratory drive index, and
wherein the cable is structured to transmit all EMG signal activity sensed by the circuitry to the controller.

14. The monitoring system of claim 13,
wherein the socket is configured to receive the cable via a slide mechanism.

15. The monitoring system of claim 13,
wherein the circuitry comprises a plurality of electrodes, each of the plurality of electrodes being electrically isolated from every other one of the plurality of electrodes.
